(19) United States
(12) Patent Application Publication
(10) Pub. No.: US 2002/0132260 A1
(43) Pub. Date: Sep. 19, 2002

(54) Quantitative immunohistochemistry (QIHC)

(76) Inventors: Mark G. Erlander, Encinitas, CA (US); Ranelle C. Salunga, San Diego, CA (US); Kimberly Stecker, Carlsbad, CA (US); James L. Wittliff, Louisville, KY (US)

Correspondence Address: Kawai Lau, Morrison & Foerster LLP, 3811 Valley Centre Drive, Suite 500, San Diego, CA, 92130-2332, US

(21) Appl. No.: 10/080,435

(22) Filed: Feb. 22, 2002

Related U.S. Application Data
(60) Provisional application No. 60/271,344, filed on Feb. 22, 2001.
(60) Provisional application No. 60/314,697, filed on Aug. 23, 2001.

(51) Int. Cl. C12Q 1/68; G01N 33/53; G01N 33/567

(52) U.S. Cl. 435/6; 435/7.2

(57) ABSTRACT

The present invention is directed to methods and compositions for the detection of a ligand in a cell containing sample by contact with a binding agent capable of binding said ligand, isolating cells from said sample, and detecting said ligand by detection of a nucleic acid marker conjugated to said binding agent. The invention may be practiced either quantitatively or qualitatively. Detection of ligand from a sample containing as few as one to two cells is described.

### RELATED APPLICATIONS

This application claims benefit of priority from U.S. Provisional Patent Applications No. 60/271,344, filed Feb. 22, 2001, and 60/314,697, filed Aug. 23, 2001, which are hereby incorporated by reference in their entireties as if fully set forth.

### TECHNICAL FIELD

The present invention relates to methods and compositions for detection and/or identification of a ligand in a cell and/or tissue sample. The present invention utilizes a binding agent capable of binding to said ligand, a means of detecting said binding agent when bound to the ligand, and a means of isolating said cell and/or tissue to permit detection and quantitation of the ligand by detection of said binding agent. Detection may be accomplished either quantitatively or qualitatively. Detection of ligand from a sample of as few as one to two cells is also provided.

### BACKGROUND ART

Antibodies provide a powerful and versatile means of detecting small amounts of an antigen within a sample and have been used extensively for both research and diagnostic applications. Antibodies can be produced which bind specifically to a desired antigen. A number of schemes have been created for detecting the bound antibody including the popular enzyme linked immuno-sorbent-assay (ELISA) techniques (Harlow, et al. Antibodies—A Laboratory Manual. Cold Spring Harbor Laboratory (1988)). One method of ELISA involves exposing the antigen specific antibody to an antigen bound to a substrate. The antibody-antigen complex is exposed to an anti-IgG-Peroxidase complex. A chromogen is added which is oxidized to form a colored product of the peroxidase enzyme, the amount of color being proportional to the amount of peroxidase present which is in turn related to the amount of antigen present. Another ELISA method uses a sandwich assay technique in which a capture enzyme bound to a surface is exposed to an antigen containing solution. A second antibody-enzyme complex in which the antibody portion is also directed toward the antigen is added, and this sandwich complex is detected using a chromogen activated by the enzyme.

While extremely useful, the ELISA methods have limitations in sensitivity and are not easily quantitated, especially at low levels of antigens.

Immuno-PCR is a method described by Sano, et. al. (Science, 1992, 258:120-122) to improve the sensitivity of detection by combining the specificity of antibody detection with amplification power of PCR.

Sano et. al. (U.S. Pat. No. 5,665,539) describe the detection of bovine serum albumin (BSA) protein with a monoclonal antibody. A protein A-Streptavidin chimera was then put into contact with the antibody-BSA complex such that the protein A bound specifically to the antibody. A biotinylated linear plasmid DNA (pUC19, 2.67 kb) was used to connect DNA via the streptavidin-biotin interaction. The presence of the plasmid DNA was detected with PCR with the use of primers specific for amplifying a portion of the plasmid DNA. The authors suggest that an improvement in the background could be achieved by pre-conjugation of the nucleic acid marker to the antibody.

This approach has been used in the form of a sandwich immuno-PCR assay for the simultaneous detection of multiple analytes for human thyroid-stimulating hormone: (hTSH), human chorionic gonadotrophin (hCG), and beta-galactosidase (Nucleic Acids Res 1995;23, 522-529). Each of the DNA oligonucleotide labels contained the same primer sequences for simultaneous amplification of all three by a single primer pair.

Ebersole et. al. described an improvement in immuno-PCR which uses direct attachment of the DNA label to the antibody via a chemical crosslinking agent (Clinical Chemistry, 1995, 41(9):1371-1377). They describe covalent attachment either to —SH or to —NH₂ sites on the antibody. They also describe the use of either single stranded or double stranded DNA markers covalently attached. The authors looked at a variety of attachment and PCR systems and conclude that “antibody affinity and non-specific binding of the conjugates are more important in affecting assay detection limit than the efficiency of DNA label amplification.” They state that the problem of non-specific binding can compromise performance because of higher than desired background signals.

Eberwine and Rogers (U.S. Pat. No. 5,922,553) described a similar sandwich assay in which the amplification is performed with RNA amplification rather than PCR. In this method, a double-stranded DNA marker containing a promoter sequence for an RNA polymerase enzyme such as T7 polymerase is attached to an antibody. They also note that while PCR can be used as described by Sano et al. to amplify a marker sequence and thus detect a protein, “this amplification is not quantitative for >10-fold differences in antigen concentration. Thus, there is no direct correlation between the amount of signal and the amount of protein present” (see column 2, lines 21-26).

Cao et. al. describe a method in which the immuno-PCR is performed in-situ and detected by hybridization (Lancet 356, 1002-1003 (2000)) Shweitzer et al. utilize rolling circle as the method of amplification (Proceedings Of The National Academy Of Sciences Of The United States Of America 97, 10113-10119 (2000)). Sims et. al. use “real-time” PCT for detection with the immuno-PCR process and to improve quantitation of the PCR amplified DNA (Analytical Biochemistry 281, 230-232 (2000)). Zhang et. al. showed that immuno-PCR could be used for detecting cell surface carbohydrate tumor antigens on tumor cells in blood (American Journal Of Pathology, 152, 1427-1432 (1998)).

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### DISCLOSURE OF THE INVENTION

The present invention provides a system to detect and/or identify a ligand in a cell and/or tissue sample. The invention utilizes a binding agent, conjugated to a marker and capable of binding to said ligand. Detection of the presence of the marker of a binding agent bound to the sample indicates the presence of the ligand. Isolation of binding agent bound cells from the sample prior to detection of the marker provides the present invention with advantages over previously used “immuno-PCR” techniques, where non-specific background signals posed a significant problem.

In one aspect of the invention, isolating or capturing the binding agent bound cells may be performed by the use of microdissection such that only a small portion of cells or tissues from a sample are used to detect the presence of the marker. The selection of specific cells or regions of tissue has been found to surprisingly result in extremely low levels of background signal without the need for a “sandwich” type assay where a ligand is first immobilized based on binding to a first antibody, washed, and then bound to a second (marker linked) antibody for detection (see column 3, line 50 to column 4, line 4 of U.S. Pat. No. 5,922,553). The low levels of background signal seen with isolated or captured cells provide an increased ability to detect and quantitate ligands, especially those which are present at low levels. Without being bound by theory, this ability may be due to the very high signal to noise measurements observed with the present invention.

In one feature of the invention, the binding agent is an antibody, and the ligand is an antigen recognized and bound by the antibody. In another feature of the invention, the marker is a nucleic acid molecule which may be detected by direct or indirect means. In a further feature of the invention, quantitative measurement of the expressed level of a ligand, even at an extremely low concentration, is provided. This feature of the invention may also be performed in a multiplex mode such that simultaneous quantitative measurement of the levels of multiple ligands from samples such as tissue sections and cell smears can be performed.

In one embodiment of the invention, a method is provided for measuring the level of expression of specific ligands in a cell or tissue sample by

contacting the sample with a binding agent capable of binding to a ligand of interest wherein each binding agent is conjugated to a nucleic acid molecule;

isolating or capturing cell containing portions of the sample; and

detecting the nucleic acid molecule from the isolated or captured portions of the sample to indicate the presence of said ligand of interest.

In preferred embodiments, the above may be optionally practiced by the use of multiple binding agents, each conjugated to a different nucleic acid molecule and each capable of binding a different ligand or a different form of one ligand; the isolation of cell containing portions of a sample after staining the sample to identify cells of interest to isolate; the isolation of cell containing portions of the sample by microdissection; the detection of nucleic acid molecules by amplification thereof, and/or the contact between a sample and the binding agent is preceded by hydrating and non-specifically blocking the sample (with, as a non-limiting example, BSA and herring sperm DNA or Cot1 DNA or other suitable non-specific blockers for protein and DNA). Preferably, the isolated cell containing portion is processed to be a lysate or extract or chromatographed or to be another processed form prior to detection.

Alternatively, the invention may be practiced with isolation of cell containing portions of the sample prior to contact with the binding agent and detection of markers conjugated thereto. In this alternative embodiment, the isolated cell containing portions may also be processed and then assayed by a “sandwich” type method wherein the processed material is contacted with an immobilized binding agent under conditions wherein the ligand will bind said agent, washed to remove unbound material, contacted with a binding agent conjugated to a marker as described herein, and then assayed for presence of the ligand by detection of said marker.

In another embodiment of the invention which may be practiced with any of the above options, detection of covalently modified or structurally altered forms of a ligand of interest is provided. As a non-limiting example, two different ligands (antigens) may be quantitated by the use of two nucleic acid (preferably DNA) conjugated binding agents (antibodies), with one antibody-DNA could be specific to all forms of a ligand (e.g. native protein) while the second primary is specific for only the phosphorylated form of the same protein. This general method is provided as follows:

contacting tissue samples with two or more different antibodies each conjugated to a different nucleic acid molecule;

isolating or capturing cell containing portions of the sample after identification of cells of interest; and

detecting the presence of the nucleic acid molecules in the isolated or captured portions of the sample to indicate the presence of different ligands.

In other preferred embodiments, the above may be practiced with the quantitation of the fraction of a protein that is covalently modified (for example, phosphorylated) by using multiplex quantitative PCR. A multiplex quantitative PCR is run to obtain a ratio of the relative amounts of the two different nucleic acid molecules (bound by to the antibodies), which ultimately measures the fraction of protein in the modified (e.g. phosphorylated) form. As a non-limiting example, if the protein is a protein “X” that is active (e.g., enzymatically active) when phosphorylated then the fraction of protein X is in the phosphorylated form would be the quotient of [amount of phosphorylated protein X]/[total amount of protein X].

Stated in terms of one preferred embodiment, a tissue sample (section) is contacted with antibodies Ab-DNA1 and Ab-DNA2 which recognize Protein X and Protein X-phosphorylated, respectively. The tissue sample is then stained (via histochemical staining such as hematoxylin and eosin) or immunostaining (such as a secondary antibody that recognizes the two primary antibodies conjugated to DNAs) followed by laser capture microdissection (LCM) or laser microdissection (LMD) of cells of interest. DNA molecules DNA1 and DNA2 can then be detected via amplification as described herein to determine the relative amounts of Protein X and Protein X-phosphorylated.

An application of this embodiment of the invention is to measure the fraction of a given enzyme that is in the modified (e.g. phosphorylated) form and thus “active”. The calculated quotient, [amount of phosphorylated protein X]/[total amount of protein X], is the “activity quotient” for that specific enzyme. As a non-limiting example, EGF-receptors (part of the tyrosine kinase receptor family) are activated (kinase enzymatic activity) by phosphorylation of tyrosine(s) residues. Activity quotients of [phosphorylated]/[total HER-2/neu](a member of the EGF receptor family) could be used to determine the fraction of HER-2/neu that is in the activated state. Recent evidence suggests that the measurement of the phosphorylated form of HER-2/neu improves the prognostic value of HER-2/neu in breast cancer. Additionally, the activity quotient may be a better predictor of response to trastuzumab, doxorubicin or tamoxifen and other cancer-related therapies. In addition to HER-2/neu, drugs are known to inhibit the kinase activity of the EGF-receptor. Thus, an activity quotient can measure the amount of “activated” EGF receptor in the presence or absence of a receptor modulator (activator or inhibitor).

This embodiment of the invention may also be applied to other covalent modifications, including, but not limited to, prenylation, glycosylation, lipidation, and covalent poly(ADP-ribosyl)ation. Of course quantitation of other covalent modifications that exist for a given protein do not necessarily change the “activity” of the protein but may change other properties of the protein, such as, but not limited to, subcellular location, ability to interact with other proteins, and stability. Any type of covalent modification of a protein that can be specifically identified and bound by a binding agent conjugated to a specific NA sequence may be assayed by the present invention.

This embodiment of the invention may also be applied to identifying structurally altered proteins. As a non-limiting example, a ligand binding to its respective receptor usually induces a structural change in the receptor. This “change” may result in events such as (but not limited to) dimerization at the cell surface (e.g., tyrosine kinase receptors such as those of the EGF receptor family) or passage of the receptor into the nucleus (e.g., estrogen receptor/steroid receptor family). Use of unique antibodies tagged with unique nucleic acid molecules and capable of recognizing different protein (receptor) forms and/or locations permits the present invention to provide quantitation (as described above for phosphorylated states) of the fraction of a given protein in a particular 3-dimensional form or physical location.

Furthermore, different types of ratios or quotients may be measured and thus calculated between two or more different covalent forms of the same protein. Thus the invention is not restricted to using only all protein forms of a given protein as a denominator in ratio/quotient measurements and subsequent calculations. As a non-limiting example, a protein that is both glycosylated and phosphorylated may be analyzed by using a ratio of [glycosylated]/[glycosylated+phosphorylated] such that the amount of unmodified forms of the protein are excluded from the denominator. Similarly, the numerator may include the amount of more than one modified form of a protein. Thus ratios such as [glycosylated+phosphorylated]/[glycosylated+phosphorylated+unmodified] may also be used as deemed appropriate by the skilled person.

### BRIEF DESCRIPTION OF THE DRAWINGS

FIG. 1 is a schematic representation of one embodiment of the invention in a multiplex mode. Five binding agents conjugated to nucleic acid molecules A, B, C, D, and E (preferably primary antibodies conjugated to five different DNA molecules) are bound to five different cellular ligands in a cell containing sample. Desired cells are captured followed by amplification of the nucleic acid molecules by quantitative PCR or for hybridization to an array (preferably a microarray) of nucleic acid molecules. While this representation highlights the detection of cellular ligands on the cell surface, the invention is by no means so limited.

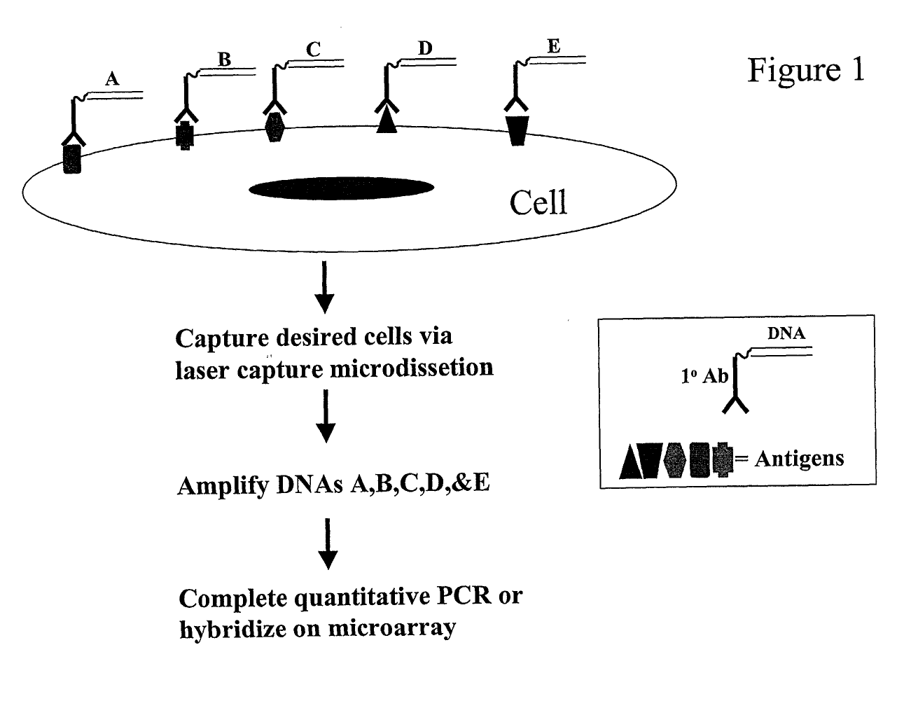

FIG. 2A shows the detection of prostate specific antigen (PSA) in laser captured human epithelial and stromal cells (approximately 500 for each sample) by use of the present invention.

FIG. 2B shows the use results of using the present invention to detect prostate specific antigen (PSA) in laser captured human epithelial and stromal cells (approximately 1-2 cells for each sample).

### MODES OF CARRYING OUT THE INVENTION

Definitions:

As used herein, “detect” or “detecting” a ligand of a cell refers to finding or discovering the presence or existence of said ligand. Use of “identify” or “identifying” with respect to a ligand of a cell refers to determining the identity of said ligand by use of a binding agent that recognizes and binds to the ligand.

The term “ligand” of a cell refers to a component of a cell, including, but not limited to molecules found in or on, produced by, or secreted by a cell. The term encompasses proteins (polypeptides and/or peptides) found in, on or outside a cell as well as a biomolecule such as nucleic acids, lipids, carbohydrates, metabolites, and combinations thereof. In the case of proteins, the term includes proteins of any size and conformation, including multimeric proteins, that may be detected by a binding agent. The term also encompasses a portion or domain or epitope of any protein or other biomolecule that may be detected by a binding agent.

The terms “cell” and/or “tissue sample” includes, but is not limited to, a tissue section, a cytospin, or a cell smear. The terms encompass samples regardless of their physical condition; stated differently, the terms do not exclude material by virtue of the physical state (such as, but not limited to, being frozen or stained or otherwise treated).

The sample is usually a thin sample, less than 50 microns thick, usually less than 10 microns thick. The sample is of biological origin, usually of eucaryotic organisms, but could be that of a prokaryotic organism as well. In preferred embodiments, the sample is a human sample, but animal or plant samples may also be used in the practice of the invention. Non-limiting sources of a sample for use in the present invention include solid tissue, fluidic extracts, blood, plasma, serum, spinal fluid, lymph fluid, the external sections of the skin, respiratory, intestinal, and genitourinary tracts, tears, saliva, milk, tumors, organs, cell cultures or cell culture constituents. The present invention is particularly useful for solid tissue samples where the amount of available material is small. The method can be used to examine an aspect of expression of a ligand or a state of a sample, including, but not limited to, comparing different types of cells or tissues, comparing different developmental stages, and detecting or determining the presence and or type of disease or abnormality.

The term “binding agent” refers to any molecule capable of binding a ligand of the invention under suitable conditions. Preferably, the binding occurs with sufficient specificity to exclude detectable binding to more than one other ligand. Even more preferred are “binding agents” that bind with specific specificity to one ligand such that no detectable binding is observed for other ligands. In preferred embodiments of the invention, the “binding agent” is an antibody or ligand binding fragment or analog thereof. The “binding agent” may also be other proteins or nucleic acids, or portions or analogs thereof, that bind a ligand in the practice of the present invention. A “binding agent” and its cognate ligand may be considered binding pairs, of which non-limiting examples include receptors and their ligands; enzymes and substrates or substrate analogs or pseudo-substrates (substrate analogs that cannot be catalyzed by the enzymatic activity); enzymes and their cofactors or inhibitors or modulators; and nucleic acid and nucleic acid binding proteins. Methods for the generation of antibodies as binding agents are known in the art.

In the practice of the invention, binding agent that remains unbound to ligand of a sample after contact therewith may of course be washed away prior to further treatment(s) of the sample.

The present invention can be practiced with any molecule that can identify a specific ligand (e.g. protein as a non-limiting example) and its covalent/structural form provided that the molecule is attached or conjugated to distinct nucleic acid sequences attached to it. Appropriate interactions (such as protein-protein or protein-nucleic acid interactions) between the specific target protein (ligand) to be identified and the binding agent (molecule capable of linkage to a nucleic acid molecule) is sufficient to support use of the binding agent in the present invention.

As used herein, “conjugated” or “attached” refer to the covalent or non-covalent, as well as direct or indirect, association of a binding agent and a nucleic acid molecule. In preferred embodiments, the nucleic acid molecule is connected to the binding agent covalently, but it may also be attached non-covalently, via, for example, protein-protein interactions (such as, but not limited to, biotin/streptavidin or protein A/antibody interactions) or complexation.

Practice of the present invention with the use of an indirect association of a binding agent to a nucleic acid molecule may be exemplified as follows: contacting a ligand with a binding agent to form a first complex; contacting the first complex with a linker molecule capable of binding both the binding agent and biotin to form a second complex; contacting the second complex with a biotinylated nucleic acid molecule to form a third complex; detecting the complexed nucleic acid molecule after amplification thereof. As will be appreciated by those skilled in the art, the above acts may be performed in other sequences or combinations, such as, but not limited to, forming a complex of the linker molecule and a biotinylated nucleic acid molecule before contacting the complex with the first complex described above. The skilled artisan would also appreciate that where an antibody is used as the binding agent, one example of a linker molecule would be a streptavidin-protein A chimeric (fusion) protein wherein the streptavidin would bind the biotinylated nucleic acid molecule while the protein A would bind the antibody.

The above may also be modified to be practiced with a less indirect association between a binding agent and a nucleic acid molecule. As a non-limiting example, the streptavidin may be covalently attached to the binding agent such that no linker molecule is necessary because the biotinylated nucleic can bind to the binding agent through the binding interactions between streptavidin and biotin.

In preferred embodiments of the invention, the nucleic acid molecule is selected to provide a unique tag for the binding agent to which it is attached. The nucleic acid molecule may thus be termed a “nucleic acid tag” or “NA-tag” on the binding agent. The nucleic acid molecule may contain regions which can be used for amplification by PCR (the polymerase chain reaction), such as by inclusion of specific primer regions are included within the molecule. The specific primer regions may be selected to be identical or different between different nucleic acid molecules attached to more than one binding agent. The nucleic acid molecule can be either single or double stranded, and is between about 10 and 1000 nucleotides in length, preferably between about 30 and about 200 or about 300 nucleotides in length. Also preferred are molecules about 200, about 300, about 400, about 500, about 600, about 700, about 800, or about 900 in length.

The use of different nucleic acid molecules conjugated to different binding agents permit detection of the different ligands bound by said agents. In one embodiment of this aspect of the invention, the different nucleic acid molecules contain different sequences which may be amplified by use of the same primers. In another embodiment, the different nucleic acid molecules contain different sequences which are amplified by use of different primers. In preferred embodiments of the invention, the length of the sequences (of different nucleic acid molecules) amplified by PCR are identical, or nearly identical, in length.

Alternatively, the nucleic acid molecule may contain a promoter region. Any RNA polymerase promoter region may be used. Suitable promoter regions will be capable of initiating transcription from an operably linked DNA sequence in the presence of ribonucleotides and an RNA polymerase under suitable conditions. The promoter region will usually comprise between about 15 and 250 nucleotides, preferably between about 17 and 60 nucleotides, from a naturally occurring RNA polymerase promoter, a consensus promoter region, or an artificial promoter region, as described in Alberts et al. (1989) in Molecular Biology of the Cell, 2d ed. (Garland Publishing, Inc.). In general, prokaryotic promoters are preferred over eukaryotic promoters, and phage or virus promoters are most preferred. As used herein, the term “operably linked” refers to a functional linkage between the affecting sequence (typically a promoter) and the controlled sequence (the mRNA binding site). The promoter sequence can be from a prokaryotic or eukaryotic source. Representative promoter regions of particular interest include T7, T3 and SP6 as described in Chamberlin and Ryan, The Enzymes (ed. P. Boyer, Academic Press, New York) (1982) pp 87-108. In a preferred embodiment, the RNA polymerase promoter sequence is a T7 RNA polymerase promoter sequence comprising at least nucleotides −17 to +6 of a wild-type T7 RNA polymerase promoter sequence, preferably joined to at least 20, preferably at least 30 nucleotides of upstream flanking sequence, particularly upstream T7 RNA polymerase promoter flanking sequence. Additional downstream flanking sequence, particularly downstream T7 RNA polymerase promoter flanking sequence, e.g. nucleotides +7 to +10, may also be advantageously used. For example, in one particular embodiment, the promoter comprises nucleotides −50 to +10 of a natural class III T7 RNA polymerase promoter sequence.

The presence of a promoter permits sequences in the nucleic acid molecule to be amplified linearly by production of transcribed RNA molecules. The promoter containing nucleic acid molecule is between about 100 and 2000 nucleotides in length, preferably between about 100 and about 200, 300, 400, 500, 600, 700, 800, or about 900 nucleotides in length. The use of different nucleic acid molecules conjugated to different binding agents permit detection of the different ligands bound by said agents upon activation of transcription of the different nucleic acids. In one embodiment of this aspect of the invention, the different nucleic acid molecules are each operably linked to a copy of the same promoter region such that the same cognate RNA polymerase may be used to transcribe all of the nucleic acid molecules. In an alternative embodiment, the different nucleic acid molecules are each operably linked to two or more different promoter regions which may be transcribed by different RNA polymerases. In preferred embodiments of the invention, the length of the transcripts of different nucleic acid molecules are identical, or nearly identical, in length. Transcription products may be detected by contacting them with a microarray comprising nucleic acid molecules capable of binding said products by base pair complementarity.

The term “marker” refers to any detectable molecule that may be conjugated to a binding agent of the invention. In preferred embodiments, it is a nucleic acid molecule. As a non-limiting example, and where the binding agent is an antibody, the marker may be a double stranded DNA molecule that is attached to the antibody using glutaraldehyde and ethanolamine for extended periods of time. As recognized by those skilled in the art, conjugated binding agents may be stored at 4 degrees Celsius or frozen until they are used.

As used herein, “microdissection” or any variation of the term broadly refers to mean separation technique by which portions of a cell containing sample can be separated from other portions of the sample. Non-limiting examples of an appropriate means to isolate some cells from a cell containing sample are laser-capture microdissection (LCM), laser microdissection (LMD), laser cutting, or manual microdissection. LCM is reviewed, for example, by Emmert-Buck et al. (Science, 1996, 274:998-1001). A method which permits the preparation of one or more than one cell of interest from a cell containing sample may be used in combination of the invention.

In some embodiments of the invention, it is useful to treat the sample prior to microdissection with reagents, stains, or dyes which enhances the visualization of the structure of the tissue and/or identity of the cells in order to improve microdissection. Stains, reagents and dyes specifically directed toward different parts of a cell, such as but not limited to, the nucleus, a membrane, or a subcellular structure, may also be used. Reagents, stains and dyes which are specific for, or otherwise differentiate between, different tissue types or cell types may also be used in the practice of the invention.

Separation of a section of a cell containing sample refers to isolating only the desired portion of the sample available for the detection step. One preferred method of separation is physical removal of the portion of the sample. In some cases, the separation can be achieved without actually removing the section by selectively allowing only the desired portion of tissue to contact the reagents which provide detection through amplification. Separation advantageously provides the ability to exclude portions of a sample containing non-specific bound binding agent.

In some cases it is desired that the selected portion of tissue is a homogeneous population of cells. In other cases it is desired that the selected portion is a region of tissue, e.g. the lumen as a non-limiting example. The selected portion can be as small as one cell or two cells, or could represent many thousands of cells. In most cases the collection of cells is important, and while the invention has been described for use in the detection of cellular components, the method may also be used for detecting non-cellular components of an organism (e.g. soluble components in the blood as a non-limiting example).

In embodiments of the invention where the “binding agent” is an antibody, the ligand may be referred to as an “antigen” which is recognized and bound by the antibody under suitable conditions.

“Nucleic acid molecule” refers to a polynucleotide or other polymeric form of nucleotides (ribonucleotides and deoxyribonucleotides) of any length. The term includes double-and single-stranded DNA and RNA as well as DNA/RNA hybrids. It also includes known types of modifications including labels known in the art, methylation, “caps”, substitution of one or more of the naturally occurring nucleotides with an analog, and intemucleotide modifications such as uncharged linkages (e.g., phosphorothioates, phosphorodithioates, etc.), appendant moieties (including proteins such as nucleases, toxins, antibodies, signal peptides, poly-L-lysine, etc.), intercalators (e.g., acridine, psoralen, etc.), chelators (e.g., metals, radioactive metals, etc.), alkylators, modified linkages (e.g., alpha anomeric nucleic acids, etc.), as well as unmodified forms of the polynucleotide. In preferred embodiments of the invention, the “nucleic acid molecule” is a DNA and may be referred to as a “DNA tag” attached to a binding agent.

Nucleic acid molecules of the invention may be detected by any direct or indirect means, including detection of molecules amplified therefrom. Amplification can be performed by techniques such as PCR or reverse transcription PCR (“RT-PCR”) or linear RNA amplification (via a promoter region located on said nucleic acid molecules). Other amplification methods such as Transcription Modulated Amplification (TMA), Branched DNA (bDNA), Ligase Chain Reaction (LCR), or thermophilic Strand Displacement Amplification (tSDA) may also be used.

In embodiments of the invention where more than one nucleic acid sequence are being amplified simultaneously, it is preferred that the amplifications of each sequence occur in a manner such that the relative levels of amplified materials are comparable and reflect the relative levels of biomolecule in the sample. In the case of PCR, this involves, for instance having priming sequences in each of the nucleotides which are the same or similar in annealing temperature and priming characteristics.

Amplified sequences can be detected directly, or the sequences can be converted in order for detection. One preferred method of detection is Quantitative PCR (Q-PCR) which can be performed either with intercalating probes such as CyberGreen or with fluorogenic reporters such as TaqMan probes. In preferred embodiments of the invention, “real-time” PCR instead of endpoint detection is used. In qualitative embodiments of the invention, the amplified sequences can be detected with gel or capillary electrophoresis, with radioactivity, chemiluminescence, mass spectrometry, or other methods.

The use of quantitative embodiments of the invention also permit the amount of ligand(s) bound by the binding agent(s) to be determined. In a further aspect of the invention, the absolute amounts per cell of a ligand may be calculated. As will be appreciated by those skilled in the art, quantitation will depend in part on determinations and/or estimations of a number of factors, including, but not limited to the stoichiometry of ligand to bound binding agent, the number of markers (e.g. nucleic acid molecules) attached to each binding agent (which may be affected by the coupling efficiency of marker to binding agent), and the detection of marker (e.g. quantitation of nucleic acid amplification). Such determinations, estimations and/or calculations require no undue experimentation because they are mere application of methods and knowledge known in the art.

One preferred embodiment for the measurement of multiple biomolecules simultaneously involves creating fluorescently labeled copies of the sequences (or sequences complementary thereto), and exposing the labeled material to a hybridization array containing sequences complementary to the labeled sequences. This embodiment may be used for the simultaneous measurement of the levels of 100s to 1000s of biomolecules within a sample.

A “microarray” is a linear or two-dimensional array of preferably discrete regions, each having a defined area, formed on the surface of a solid support. The density of the discrete regions on a microarray is determined by the total numbers of target polynucleotides to be detected on the surface of a single solid phase support, preferably at least about 50/cm², more preferably at least about 100/cm², even more preferably at least about 500/cm², and still more preferably at least about 1,000/cm². As used herein, a DNA microarray is an array of oligonucleotide primers or cDNAs placed on a chip or other surface. Since the position of each particular group of primers or cDNAs in the array is known, the identities of molecules hybridizing to the microarray can be determined based on their binding to a particular position in the microarray.

Relative amounts of multiple ligands can be quantitated by globally amplifying the DNA tags as a population either via PCR (using primers that are complementary to all DNA tags) or a RNA polymerase-based method (wherein all DNA tags contain a promoter used by an RNA polymerase) or a combination of the two methods. These globally amplified DNA tags are then subsequently labeled and hybridized to a microarray containing DNA sequences complementary to the DNA tags. This may be used to detect the expression of a ligand by detection of sequences amplified from a nucleic acid marker conjugated to a binding agent that binds said ligand. Signal intensity of hybridization is then used to determine a relative expression pattern or signature that is compared between ligands or to signals obtained from another set of captured (sampled) cells.

As used herein, a ligand present at a “low concentration” refers to a ligand that is present at concentrations such that detection by conventional ELISA methods is poor or not possible. It also refers to concentrations such that background noise during detection by conventional immuno-PCR (see for example U.S. Pat. No. 5,665,539) or “immuno RNA” (see for example U.S. Pat. No. 5,922,553) are avoided at least in part by use of the present invention, which may be practiced in part with the application or adaptation of these methods.

Without limiting the invention, sample “staining” may be performed by any means, including, but not limited to, histochemical staining (e.g. hematoxylin and eosin) and immunostaining (detection with an antibody followed by an enzymatic reaction yielding a color reaction product). Alternatively, the sample may remain unstained, and cells of interest identified by light microscopy or other similar methods known in the art.

The present invention may also be performed in “multiplex” mode such that simultaneous quantitative measurement of the levels of multiple ligands from samples can be performed. The invention thus quantitates the amount of various selected antigens with optionally cellular resolution. The invention may be practiced with a plurality of binding agents, such as about 2-5, 10 or even 100 (or more) different binding agents (such as antibodies), with each recognizing a specific antigen and attached to a unique DNA tag. A plurality of ligands may thus be simultaneously detected. Quantitating the amount of protein present via detection of the DNA tags is performed as described herein.

Application of the Invention

The present invention may be used to detect a large variety of ligands found in cell containing samples. In preferred embodiments of the invention, quantitation (optionally simultaneous quantitation) of the different biomarkers currently used in cancer and other diseases, especially, but not limited to, breast cancer diagnosis, prognosis and predictive medicine is performed. Individual tests that quantitate the receptors for estrogen, progesterone and HER2neu as well as several other protein markers (EGF-receptor, Cathepsin D, urokinase plasminogen activator (uPA), uPA receptor and PA inhibitor) are currently being used for diagnosis/prognosis and predictive medicine in breast cancer. The present invention may be used to generate a single test that quantitates a relative signature of all these biomarkers. This signature could then be compared to a database in which different signatures of these biomarkers are already correlated with diagnosis, prognosis and predictive medicine for breast cancer.

The invention may generally be applied to the detection of any ligand related to a specific cell type or disease condition for diagnostic and prognostic purposes. As non-limiting examples, the invention may be applied to the detection of proteins known to be expressed by particular cell types and/or to the detection of biomolecules associated with a particular disease.

The present invention may also be “scaled up” for use in a more comprehensive context. One form is where the invention is applied to determining different forms of the same ligand (protein X) with more than one “activated” state. Multiple antibodies can be incubated with a sample containing the different states of protein X with each antibody attached to a unique NA molecule. Separate multiplex PCRs could be performed on these samples using the same total amount of protein X present (all modified, e.g. phosphorylated, and unmodified, non-phosphorylated, states combined) as the denominator for each quotient of modified to unmodified protein X. Similarly, the present invention may be “scaled up” to provide activity quotients for more than one ligand (protein), which are examined simultaneously by using the appropriate binding agents (antibodies) conjugated to different NA tags with a sample of interest.

Kits and Compositions

Also provided by the invention are kits for use in the practice of the present invention as disclosed herein. Such kits may comprise containers, each with one or more of the various reagents (typically in concentrated form) utilized in the methods, including, for example, one or more binding agents, already attached to a marker or optionally with reagents for coupling a binding agent to a marker or nucleic acid molecule (as well as the marker itself); buffers, the appropriate nucleotide triphosphates (e.g. dATP, dCTP, dGTP, dTTP, dUTP, ATP, CTP, GTP and UTP), reverse transcriptase, DNA polymerase, RNA polymerase, and one or more sequence-specific or degenerate primers for use in detection of nucleic acid molecules by amplification; and/or reagents and instrumentation for the isolation (optionally by microdissection) to support the practice of the invention. A label or indicator describing, or a set of instructions for use of, kit components in a ligand detection method of the present invention, will also be typically included, where the instructions may be associated with a package insert and/or the packaging of the kit or the components thereof.

Compositions for the practice of the invention include reagents and reactants such as those described above.

The following examples are put forth so as to provide those of ordinary skill in the art with a complete disclosure and description of how to make and use the present invention, and are not intended to limit the scope of what the inventors regard as their invention nor are they intended to represent that the experiments below are all and only experiments performed. Efforts have been made to ensure accuracy with respect to numbers used (e.g. amounts, temperature, etc.) but some experimental errors and deviations should be accounted for. Unless indicated otherwise, parts are parts by weight, and temperature is in degrees Celsius.

### EXAMPLE

### Sample Preparation

7 μm human tissue prostate sections were pre-incubated in a PBS/BSA solution containing salmon sperm DNA as a blocking agent. A DNA-conjugated anti-PSA antibody at a 1:100 dilution (1 μg/ml) was incubated with the section for 1 hour. Unbound antibody was washed extensively and the tissue was counterstained with hematoxylin and eosin (H&E) and dehydrated. Approximately 500 cells were laser captured onto caps, and the caps were processed individually to prepare a lysate. A portion of the lysate was used for quantitative PCR analysis using synthetic DNA primers specific to the DNA conjugated to the antibody. Flanking PCR primers targeted against a portion of the DNA-conjugate were used in a Light Cycler real time quantitative PCR assay using Sybr Green for detection. For quantitation, a standard curve was generated by parallel amplification of known copy numbers of the same DNA conjugate.

### Results

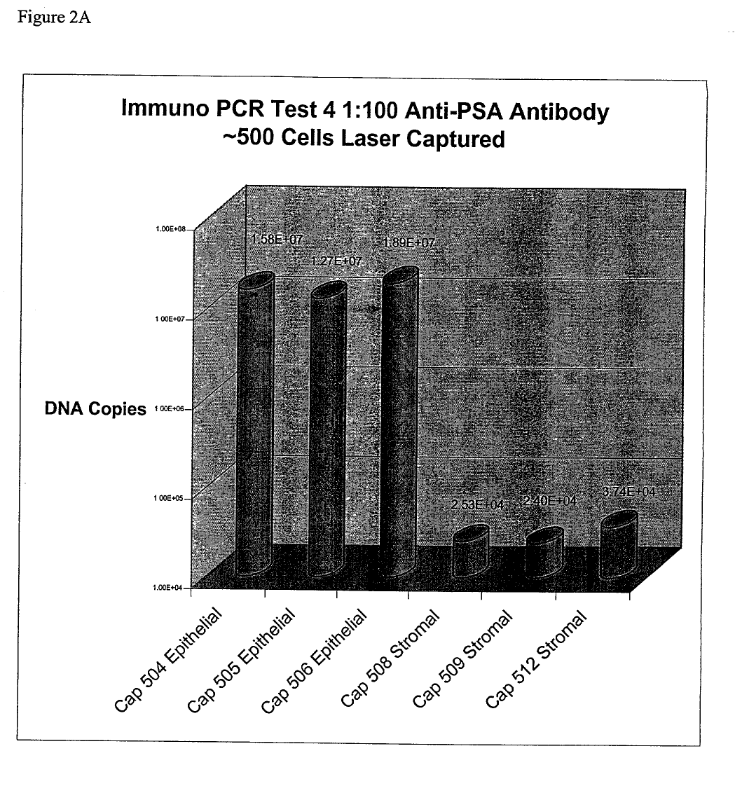

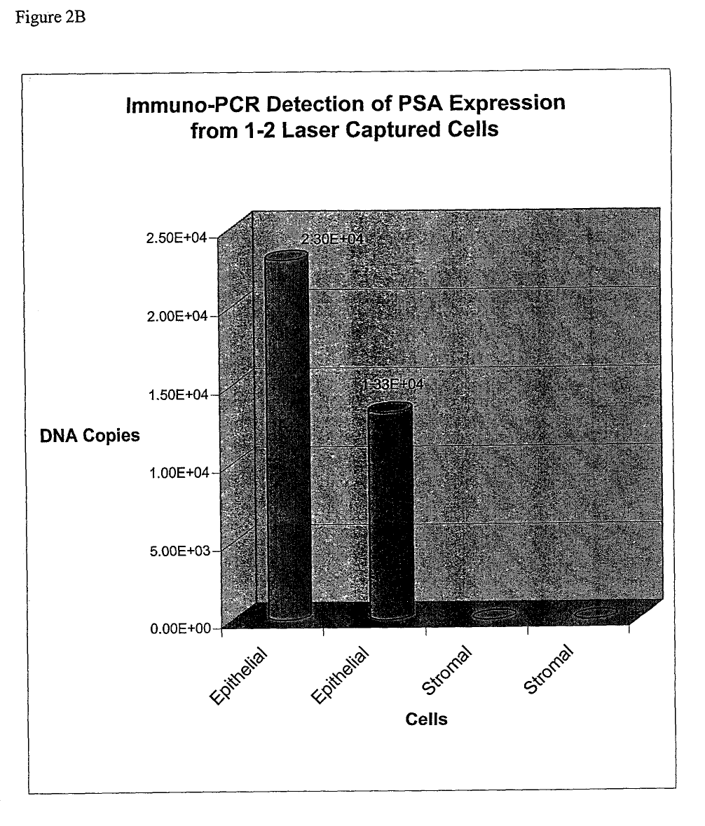

Real time quantitative PCR gave a mean DNA copy number for each set of approximately 500 stromal cells of 1.3×10⁵ copies, and a mean DNA copy number of 4.7×10⁸ copies for each set of approximately 500 epithelial cells. Results are show in FIG. 2A. This corresponds to a signal to noise ratio of 3.6×10^{3.}

This also indicates that on a per cell basis, each epithelial cell has a mean DNA copy number of approximately 9.4×10⁵. Considered in light of conjugation of approximately 3.2 DNA copies per antibody molecule and the simple approximation of one antibody molecule per PSA molecule, a value of approximately 2.9×10⁵ PSA molecules per epithelial cell can be calculated. Of course this value is an approximation and may vary somewhat depending on a number of values, including the number of antibodies bound per PSA molecule.

A similar experiment using only 1-2 captured cells for each data set (see FIG. 2B) shows detectable signal from both epithelial samples.

All references cited herein are hereby incorporated by reference in their entireties, whether previously specifically incorporated or not. As used herein, the terms “a”, “an”, and “any” are each intended to include both the singular and plural forms.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A method for detecting a ligand in a cell or tissue sample, said method comprising,
contacting said sample with a binding agent capable of binding said ligand, wherein said agent is attached to a detectable nucleic acid molecule;
staining said sample to identify cells of interest;
capturing or isolating said cells of interest; and
detecting said nucleic acid molecule
wherein the presence of said nucleic acid molecule indicates the presence of said ligand.

2. The method of claim 1 wherein said agent is an antibody.

3. The method of claim 1 wherein said sample is a tissue section, a cytospin, or a cell smear.

4. The method of claim 1 wherein said detecting is by PCR amplification of said nucleic acid molecule.

5. The method of claim 4 wherein said PCR is quantitative PCR and the quantitative presence of said ligand is detected.

6. The method of claim 1 wherein said staining is by histochemical staining.

7. The method of claim 1 wherein said capturing is laser capture microdissection (LCM) or laser microdissection (LMD).

8. The method of claim 1 wherein a plurality of agents, attached to a plurality of different nucleic acid molecules, are simultaneously used to detect a plurality of ligands.

9. The method of claim 8 wherein said agents are antibodies.

10. The method of claim 1 wherein said sample is prostate tissue.

11. The method of claim 10 wherein said ligand is prostate specific ligand.

12. The method of claim 11 wherein capturing is of only one to two cells.

13. The method of claim 1 wherein said nucleic acid molecule comprises a promoter.

14. The method of claim 13 wherein said promoter is a T7 promoter.

15. The method of claim 14 wherein said detecting comprises contacting said promoter with T7 polymerase and identifying transcription initiated from said T7 promoter.

16. The method of claim 8 wherein said plurality of ligands comprise two forms of a polypeptide.

17. The method of claim 16 wherein said two forms are the phosphorylated and unphosphorylated forms of a polypeptide.

18. The method of claim 5 further comprising quantitatively determining the amount of ligand per captured or isolated cell.

19. The method of claim 1 wherein said ligand is a polypeptide, a nucleic acid, a lipid, a carbohydrate, or a portion or domain or epitope thereof.

20 The method of claim 15 wherein said identifying is by contacting transcription products with a microarray comprising nucleic acid molecules capable of binding said products by base pair complementarity.
